(19) United States
(12) Patent Application Publication
(10) Pub. No.: US 2017/0065528 A1
(43) Pub. Date: Mar. 9, 2017

(54) Alcohol-Resistant, Dose Dumping Protective Enteric Drug Film Coating

(71) Applicant: Sensient Colors LLC, St. Louis, MO (US)

(72) Inventor: Brian Kai-ming CHENG, Chesterfield, MO (US)

(21) Appl. No.: 15/125,012

(22) PCT Filed: Mar. 3, 2015
(86) PCT No.: PCT/US2015/018499

§ 371 Date: Sep. 9, 2016

(51) Int. Cl. A61K 9/28; A61K 47/44

(52) U.S. Cl. CPC A61K 9/286; A61K 47/44; A61K 9/282

(57) ABSTRACT

The invention provides an enteric film coating comprising a salt of alginic acid and aqueous shellac, wherein the enteric film coating is alcohol resistant.

### FIELD OF THE INVENTION

The present invention relates to film coatings for enteric drugs, and specifically to alcohol resistant enteric drug film coatings which inhibit dose dumping at high alcohol concentrations in the gastrointestinal tract.

### BACKGROUND OF THE INVENTION

Dose dumping occurs when environmental factors cause the premature and/or exaggerated release of a drug. Premature and/or exaggerated release of a drug can greatly increase the concentration of a drug in the body, resulting in adverse physiological effects and, in extreme cases, drug-induced toxicity.

Dose dumping is most commonly seen with drugs taken orally, particularly oral extended-release dosage drugs, with release of the drug occurring in the gastrointestinal tract. Patients may ingest other substances, such as fatty foods or alcohol, around the same time as the drug. These other substances may act on the drug's capsule/coating to speed up drug release and/or stimulate the body's absorptive surfaces to increase the rate of drug uptake.

Drugs which exhibit a dose dumping effect when taken with alcohol are a particular problem. The alcohol content in a typical alcoholic beverage ranges from 5% to 40% by volume. To test the dose dumping effect of drugs when taken with alcohol, the Food and Drug Administration (FDA) and European Medicine Agency (EMEA) recommend using an in vitro dissolution method using 1%, 5%, 20% and 40% alcohol by volume in a pH of 1.2, 4.5 and 7 to demonstrate the release equivalency in the presence of alcohol. However, the in vitro dissolution equivalent does not translate to the in vivo bio-equivalent.

Two factors can influence the bio-absorption of the active pharmaceutical ingredient(s) (API) of a drug in the body: (1) the solubility of the coating of the drug in the presence of alcohol, and (2) the solubility of the API in the presence of alcohol. In other words, bio-absorption of an API is influenced by both a drug's coating (release of API) and the alcohol solubility of the API itself (uptake of API).

For example, for “immediate release” solid dose formulations (i.e., 75% by weight of the API is released within 15 minutes), the drug form is designed to release the API quickly, and the drug coating has little or no impact on API release or bio-absorption. The alcohol solubility of the API may, however, affect the speed at which the API is absorbed by the body. In contrast, for certain “pulse-release” formulations, such as enteric release drugs which are designed to remain stable in the acidic environment of the stomach and release the API as the drug passes to less acidic environments (i.e., small intestine), the alcohol solubility of the coating is an important feature. Therefore, both the alcohol solubility of a drug's coating and the API must be considered when designing a drug to avoid alcohol dose dumping.

When it comes to alcohol digestion, approximately 20 to 25% by volume of consumed alcohol is absorbed in the stomach with the remaining alcohol being absorbed in the small intestine. The pH of the stomach is approximately 1.2, and the pH gradually increases (becomes more alkaline) moving down the gastrointestinal (GI) tract to a pH of approximately 7 in the colon. Most alcohol is eliminated around a pH of 4.5, the approximate pH of the small intestine. The relation between alcohol concentration in the GI tract and pH is therefore also a factor to consider in designing drug coatings to prevent dose dumping in the presence of alcohol, particularly for enteric drugs (i.e., drugs in which API release is triggered by a pH of 4.5 or higher).

For example, commercial enteric drugs have a polymeric coating (i.e., methacrylate copolymers and hydroxypropylmethyl cellulose acetate succinate) designed to remain intact in the stomach (i.e., pH approximately 1.2 or lower). Once the dosage passes to the intestines (i.e., pH >4.5), enteric drug film coatings are designed to rupture or disintegrate, causing API release. However, because current commercial enteric drug polymeric coatings are alcohol soluble, the presence of alcohol in the stomach and/or small intestines will cause premature disintegration/rupture of the coating, thereby destroying the delayed-release property of the dosage and causing dose dumping.

The objective of having an alcohol resistant enteric drug film coating is to ensure there is no change in the bioavailability of an API in the presence of alcohol in the GI tract. The solubility and bio-absorption of an API can increase or decrease depending on the alcohol solubility of the API once it is released. Once the API is released, regardless of the type of release, the bioavailability is no longer an effect of the alcohol resistance of a film coating.

Current alcohol dose dumping preventative drug film coating systems are designed to release the API by controlled erosion of the coating over time. For example, ethyl cellulose is a sustained release polymer used in drug film coatings to control API release in the GI tract over time. However, ethyl cellulose containing coatings are affected by the “fed and fasting” state of the patient, and it is therefore difficult to control release times over a range of diverse patients (i.e., age, gender, body weight, etc.). The therapeutic response of patients to an ethyl cellulose coated drug is therefore unpredictable.

Solid dosage forms (i.e., tablet, pill, capsule) containing an enteric drug film coating do not exhibit any API release in the stomach (i.e., pH approximately 1.2 or lower). The alcohol resistance of an enteric drug film coating therefore should not modify API release inhibition at a pH of less than or equal to 1.2.

### SUMMARY OF THE INVENTION

In an embodiment, the invention provides an enteric drug film coating composition comprising an aqueous solution of a salt of alginic acid, preferably 1-5% by weight of a salt of alginic acid, and an aqueous shellac, the % by weight based on the total weight of the composition.

In another embodiment, the invention provides an enteric drug film coating made from the enteric drug film coating composition, wherein the enteric drug film coating is alcohol resistant.

In another embodiment, the invention provides a solid dosage form comprising a core comprising an API and an enteric drug film coating made from the enteric drug film coating composition, wherein the enteric drug film coating is alcohol resistant.

### BRIEF DESCRIPTION OF THE DRAWINGS

FIG. 1 is a graphical depiction of the data of Example 4, showing the percent release of API as a function of time of a solid dosage tablet at 4% weight gain in a pH of 1.2 for various ethanol levels as set forth in Table 5;

FIG. 2 is a graphical depiction of the data of Example 4, showing the percent release of API as a function of time of a solid dosage tablet at 5% weight gain in a pH of 1.2 for various ethanol levels as set forth in Table 5;

FIG. 3 is a graphical depiction of the data of Example 4, showing the percent release of API as a function of time of a solid dosage tablet at 4% weight gain in a pH of 4.5 for various ethanol levels as set forth in Table 6;

FIG. 4 is a graphical depiction of the data of Example 4, showing the percent release of API as a function of time of a solid dosage tablet at 5% weight gain in a pH of 4.5 for various ethanol levels as set forth in Table 6;

FIG. 5 is a graphical depiction of the data of Example 4, showing the percent release of API as a function of time of a solid dosage tablet at 4% weight gain in a pH of 7.0 for various ethanol levels as set forth in Table 7;

FIG. 6 is a graphical depiction of the data of Example 4, showing the percent release of API as a function of time of a solid dosage tablet at 5% weight gain in a pH of 7.0 for various ethanol levels as set forth in Table 7;

FIG. 7 is a graphical depiction showing the percent release of API as a function of time and pH for bio-simulation 1 as set forth in Table 8;

FIG. 8 is a graphical depiction showing the percent release of API as a function of time and ethanol content for bio-simulation 1 as set forth in Table 8;

FIG. 9 is a graphical depiction showing the percent release of API as a function time and pH for bio-simulation 2 as set forth in Table 9; and

FIG. 10 is a graphical depiction showing the percent release of API as a function of time and ethanol content for bio-simulation 2 as set forth in Table 9.

### DETAILED DESCRIPTION OF THE PREFERRED EMBODIMENT

In one embodiment the invention is an enteric drug film coating composition comprising a salt of alginic acid and an aqueous shellac in an aqueous solution.

An inventive enteric drug film coating composition may comprise a combination of two or more embodiments described herein.

### Salt of Alginic Acid Component

In an embodiment, the inventive enteric drug film coating composition is an aqueous solution comprising a salt of alginic acid. Exemplary salts of alginic acid include, but are not limited to, sodium alginate, potassium alginate, magnesium alginate, lithium alginate, ammonium alginate and combinations thereof. Preferably, the salt of alginic acid is selected from the group consisting of sodium alginate, potassium alginate and combinations thereof. More preferably, the salt of alginic acid is sodium alginate.

In one embodiment, the salt of alginic acid has a viscosity of no greater than 720 centipoise (cps) in a 1% aqueous solution of the salt, the viscosity measured as a Brookfield viscosity at ambient temperature. Preferably, the salt of alginic acid has a viscosity of less than or equal to 720 cps, or less than or equal to 450 cps, or less than or equal to 300 cps in a 1% aqueous solution of the salt. More preferably, the salt of alginic acid is a low viscosity grade of alginate having a viscosity of less than or equal to 100 cps, or less than or equal to 50 cps, or less than or equal to 25 cps in a 1% aqueous solution of the salt. Most preferably, the salt of alginic acid is a low viscosity grade of alginate having a viscosity of less than or equal to 10 cps, or less than or equal to 8 cps, or less than or equal to 6 cps, or less than or equal to 4 cps in a 1% aqueous solution of the salt.

In a preferred embodiment, the salt of alginic acid is a low viscosity grade of sodium alginate having a Brookfield viscosity at ambient temperature of less than or equal to 6 cps in a 1% aqueous solution of the salt.

In a preferred embodiment, the salt of alginic acid is a low viscosity grade of sodium alginate having a Brookfield viscosity at ambient temperature of less than or equal to 4 cps in a 1% aqueous solution of the salt.

A salt of alginic acid used in the inventive enteric film coating composition may comprise one or more embodiments described herein.

### Aqueous Shellac Component

In an embodiment, the inventive enteric drug film coating composition also comprises an aqueous shellac component.

Shellac is a resin exudate of the lac insect. Shellac is naturally insoluble in water and soluble in organic solvents, including ethanol. Shellac is generally insoluble in acidic conditions (i.e., pH from 1 to 4.5) and soluble at higher pH levels (i.e., pH from 4.5 to 14).

In an embodiment, the shellac component is in aqueous form and, preferably, in aqueous alkali salt form. In embodiments, the aqueous shellac component comprises a 5 to 35 wt % aqueous solution of an alkali salt of shellac. Exemplary alkali salts of shellac include, but are not limited to, potassium, ammonium, sodium, magnesium, alkyl ammonium and aryl ammonium salts of shellac, and combinations thereof. Preferably, the salt of shellac is ammonium salt of shellac.

### Additives

In embodiments, the inventive enteric drug film coating composition includes at least one pharmaceutical additive used in the preparation of tablets, capsules, and other orally administrable forms, which is nontoxic and compatible with other components of the composition. Suitable additives for use in enteric drug coatings, and the amounts of such additives used in enteric drug coatings, are known and conventionally used in the art.

In one embodiment, exemplary additives for use in the inventive enteric drug film coating compositions are selected from the group consisting of a detackifier, a slip aid, a surfactant, a plasticizer, a preservative, an opacifier, a colorant, and combinations thereof. Preferably, the inventive enteric drug film coating composition includes at least one additive selected from the group consisting of a detackifier, slip aid, surfactant, plasticizer, and combinations thereof.

In one embodiment, the inventive enteric drug film coating composition includes at least one detackifier, at least one slip aid, at least one surfactant, at least one plasticizer and, optionally, at least one further additive selected from the group consisting of a preservative, an opacifier, a colorant, and combinations thereof.

In one embodiment, the inventive enteric drug film coating composition includes at least one detackifier. Suitable detackifiers are known in the art. Exemplary detackifiers include, but are not limited to, silicon dioxide, stearic acid, salts of fatty acids, talc, calcium chloride, dicalcium phosphate, starch, maltodextrin, lactose, microcrystalline cellulose, mannitol, xylitol, sorbitol, maltitol, and combinations thereof. Preferably, the detackifier is selected from the group consisting of silicon dioxide, salt of fatty acids, talc and stearic acid. More preferably, the detackifier is stearic acid.

In one embodiment, the inventive enteric drug film coating composition includes at least one slip aid. Exemplary slip aids include, but are not limited to stearic acid, magnesium stearate, silicon dioxide, talc, and combinations thereof.

In one embodiment, the inventive enteric drug film coating composition includes at least one surfactant. Exemplary surfactants include, but are not limited to, lecithin, polysorbates, glycerol esters of fatty acids, sugar esters of fatty acids, and combinations thereof.

In one embodiment, the surfactant is selected from the group consisting of polysorbate 80, lecithin, and combinations thereof.

In one embodiment, the surfactant is polysorbate 80.

In one embodiment, the surfactant is lecithin.

In one embodiment, the surfactant comprises polysorbate 80 and lecithin.

In one embodiment, the inventive enteric drug film coating composition includes at least one plasticizer. Exemplary plasticizers include, but are not limited to, glycerin, acetylated glyceride, medium chain triglycerides (e.g., 8-12 carbons long), lecithin, naturally-derived oils (i.e., oils from vegetable sources), mineral oils, triacetin, triethyl citrate, propylene glycol, polyethylene glycol, dibutyl sebacate, and combinations thereof.

In one embodiment, the plasticizer is selected from the group consisting of glycerin, acetylated monoglycerides, medium chain triglycerides, and combinations thereof.

In one embodiment, the plasticizer comprises glycerin, acetylated monoglycerides, medium chain triglycerides, and combinations thereof.

In one embodiment, the plasticizer comprises glycerin and acetylated monoglycerides.

In one embodiment, the inventive enteric drug film coating composition may optionally include an additional additive or additives selected from the group consisting of preservatives, opacifiers, colorants, and combinations thereof. Suitable preservatives, opacifiers and colorants for use with enteric drug film coating compositions are known in the art.

In one embodiment, the inventive enteric drug film coating composition includes a preservative. Exemplary preservatives include, but are not limited to, salts of sorbic acid, slats of benzoic acid, methyl parabens, propyl parabens, and combinations thereof.

In one embodiment, the inventive enteric drug film coating composition includes a opacifier. Exemplary opacifiers include, but are not limited to, titanium dioxide, zinc oxide, calcium diphosphate, and combinations thereof.

In one embodiment, the inventive enteric drug film coating composition includes a colorant. Exemplary colorants include, but are not limited to, synthetic dyes, lakes, naturally-derived colors, oxides, and combinations thereof.

### Enteric Drug Film Coating Composition

In one embodiment, the inventive enteric drug film coating composition is an aqueous solution comprising a salt of alginic acid and an aqueous shellac component as described herein.

In one embodiment, the inventive enteric drug film coating composition is an aqueous solution comprising less than 10% by weight of a salt of alginic acid as described herein. Preferably, the inventive enteric drug film coating composition is an aqueous solution comprising less than or equal to 5%, or from 1% to 5%, or from 3% to 4%, by weight, of a salt of alginic acid.

In one embodiment, the inventive enteric drug film coating composition is an aqueous solution comprising less than or equal to 5% by weight of sodium alginate. In a preferred embodiment, the inventive enteric drug coating composition is an aqueous solution comprising from 1% to 5% by weight of sodium alginate. More preferably, the inventive enteric drug film coating composition is an aqueous solution comprising from 3% to 4% by weight of sodium alginate.

In one embodiment, the inventive enteric drug film coating composition is an aqueous solution comprising at most 50% by weight and at least 10% by weight of an alkali salt of shellac, as described herein. Preferably, the inventive enteric drug film coating composition is an aqueous solution comprising less than 15%, or less than or equal to 20%, or less than or equal to 45%, by weight, and at least 35%, or at least 30%, or at least 25%, by weight, of an alkali salt of shellac. More preferably, the enteric drug film coating composition comprises from 3 to 40% by weight of an alkali salt of shellac.

In one embodiment, the inventive enteric drug film coating composition is an aqueous solution comprising at most 50% by weight of an ammonium salt of shellac. In a preferred embodiment, the inventive enteric drug film coating composition is an aqueous solution comprising less than 50% by weight of ammonium salt of shellac. More preferably, the inventive enteric drug film coating composition is an aqueous solution comprising less than or equal to 45% by weight of ammonium salt of shellac. In an embodiment, the enteric drug film coating composition is an aqueous solution comprising at least 50%, preferably at least 45%, and more preferably at least 40% by weight of ammonium salt of shellac.

In one embodiment, the inventive enteric drug film coating composition optionally comprises at least one pharmaceutical additive selected from the group consisting of a detackifier, a slip aid, a surfactant, plasticizer, preservative, opacifier, colorant, and combinations thereof. The total amount of additive in an enteric drug film coating composition may vary based on the intended API used with the coating, desired properties of the coating, the type and size of the dosage form (i.e., tablet, soft gel, two-piece hard capsule), among other factors that are conventional and understood in the art.

In one embodiment, the inventive enteric drug film coating composition consists essentially of an aqueous solution of a salt of alginic acid and an aqueous salt of shellac. In further embodiments, the inventive enteric film coating consists essentially of an aqueous solution of a salt of alginic acid, an aqueous salt of shellac and, optionally, one or more additives as described herein. In one embodiment, the inventive enteric drug film coating composition consists of an aqueous solution of a salt of alginic acid and an aqueous salt of shellac.

### Enteric Drug Film Coating

In one embodiment, the invention is an enteric drug film coating made from a composition comprising an aqueous solution of a salt of alginic acid and an aqueous shellac component, as described herein, wherein the enteric drug film coating is alcohol resistant.

An inventive enteric drug film coating may comprise a combination of two or more embodiments described herein.

Enteric drug film coatings do not rupture or disintegrate in the stomach (i.e., pH of 1.2 or less), and solid dosage forms containing an enteric drug film coating therefore do not exhibit any API release or has limited API release over 2 hours in the stomach. The alcohol resistance of the enteric drug film coating therefore should not modify API release inhibition at pH 1.2. Enteric drug film coatings are specifically designed to rupture and/or disintegrate in the intestines (i.e., pH >4.5). Therefore, the enteric drug film coating at this stage does not affect the bioavailability, but rather is affected by the API bio-absorption in the presence of alcohol. Alcohol resistant enteric drug film coatings resist rupture/disintegration in the presence of significant amounts of alcohol, resulting in little to no API release in the small intestine (i.e., pH >4.5), despite the appropriate pH.

The inventive enteric drug film coating accounts for the solubility of both the coating and an API. The inventive enteric drug film coating also ruptures and/or disintegrates only under low alcohol conditions (i.e., less than 30%, or preferably less than 25%, or preferably less than 5% by volume ethanol) and at a high pH (i.e., greater than 1.5, or preferably greater than or equal to 4.5).

As used herein, the term “alcohol resistant” means that disintegration and/or rupture of an inventive enteric drug film coating (and the subsequent release of an API) is prevented or limited in environments having a pH greater than 1.2 and an alcohol content of greater than 5% by volume ethanol.

In one embodiment, the inventive enteric drug film coating is alcohol resistant such that less than 30% by weight of the total weight of the API contained in a solid dosage form coated with the enteric drug film coating, is released after exposure of the solid dosage form to an environment having a pH greater than 1.2 and an alcohol content of greater than 25% by volume ethanol for up to 30 minutes, or up to 60 minutes, or up to 90 minutes, or up to 120 minutes.

In one embodiment, the inventive enteric drug film coating is alcohol resistant such that less than 30%, or less than 25%, or less than 20%, by weight, of the total weight of the API contained in a solid dosage form coated with the enteric drug film coating, is released after exposure of the solid dosage form to an environment having a pH greater than 1.2 and an alcohol content of greater than 25% by volume ethanol for up to 30 minutes, or up to 60 minutes, or up to 90 minutes, or up to 120 minutes.

In one embodiment, the inventive enteric drug film coating is alcohol resistant such that less than 30%, or less than 25%, or less than 20%, by weight, of the total weight of the API contained in a solid dosage form coated with the enteric drug film coating, is released after exposure of the solid dosage form to an environment having a pH of greater than or equal to 4.5, or greater than or equal to 7.0, and an alcohol content of greater than 25% by volume ethanol for up to 30 minutes, or up to 60 minutes, or up to 90 minutes, or up to 120 minutes.

In one embodiment, the inventive enteric drug film coating is alcohol resistant such that less than 30% by weight of the total weight of the API contained in a solid dosage form coated with the enteric drug film coating, is released after exposure of the solid dosage form to an environment having a pH of greater than 1.2 and an alcohol content of greater than 25%, or greater than or equal to 30%, or greater than or equal to 35%, or greater than or equal to 40%, by volume, ethanol for up to 30 minutes, or up to 60 minutes, or up to 90 minutes, or up to 120 minutes.

In one embodiment, the inventive enteric drug film coating is alcohol resistant such that less than 30%, or less than 25%, or less than 20%, by weight, of the total weight of the API contained in a solid dosage form coated with the enteric drug film coating, is released after exposure of the solid dosage form to an environment having a pH of greater than 1.2 and an alcohol content of greater than 25%, or greater than or equal to 30%, or greater than or equal to 35%, or greater than or equal to 40%, by volume, ethanol for up to 30 minutes, or up to 60 minutes, or up to 90 minutes, or up to 120 minutes.

In one embodiment, the inventive enteric drug film coating is alcohol resistant such that less than 30%, or less than 25%, or less than 20%, by weight, of the total weight of the API contained in a solid dosage form coated with the enteric drug film coating, is released after exposure of the solid dosage form to an environment having a pH of greater than or equal to 4.5, or greater than or equal to 7.0, and an alcohol content of greater than 25%, or greater than or equal to 30%, or greater than or equal to 35%, or greater than or equal to 40%, by volume, ethanol for up to 30 minutes, or up to 60 minutes, or up to 90 minutes, or up to 120 minutes.

In a preferred embodiment, the inventive enteric drug film coating is alcohol resistant such that less than 30%, or less than 25%, or less than 20%, by weight, of the total weight of the API in a solid dosage form coated with the enteric drug film coating, is released after the solid dosage form is exposed to at least one, and preferably both, of the following conditions for at least 60 minutes:

(i) pH=4.5, ethanol content 40% by volume; and

(ii) pH=7.0, ethanol content 40% by volume.

### Solid Dosage Form

In an embodiment, the invention is an oral solid dosage form having a controlled release profile for release of an API within the intestinal tract, the solid dosage form comprising a core comprising the API and a coating comprising the enteric drug film coating composition as described herein. In one embodiment, the invention is a solid dosage form of a drug (i.e., tablets, pill, capsule, etc.) comprising a core comprising an API and an alcohol resistant, enteric drug film coating. In embodiments, the solid dosage form comprises an alcohol resistant, enteric drug film coating made from the enteric drug film coating composition described herein.

An inventive solid dosage form may comprise a combination of two or more embodiments described herein.

In a preferred embodiment, the core comprises an API and is a core such as those known in the art.

In embodiments, the alcohol resistant, enteric drug film coating substantially surrounds the API-containing core. In preferred embodiments, the alcohol resistant, enteric drug film coating entirely surrounds the API-containing core. The thickness of the coating is typically correlated with coating weight gain as the end point determination.

In one embodiment, the coating weight gain of the solid dosage form after coating with the alcohol resistant, enteric drug film coating is less than or equal to 15% by weight of the total weight of the solid dosage form. Preferably, the coating weight gain of the solid dosage form after coating with the alcohol resistant, enteric drug film coating is less than 15%, or less than or equal to 10%, by weight, of the total weight of the solid dosage form. More preferably, the coating weight gain of the solid dosage form after coating with the alcohol resistant, enteric drug film coating is from 1 to 10%, or from 3% to 7%, or from 3.5% to 5%, by weight, of the total weight of the solid dosage form.

In one embodiment, the inventive enteric drug film coating is the only coating on the core of the solid dosage form. In a further embodiment, additional coatings may be included with the inventive enteric drug film coating. Additional coatings may be on top of the enteric drug film coating, below the enteric drug film coating, or both. For example, additional coatings may include, but are not limited to, subcoatings between the core and inventive enteric drug film coating and top coatings applied on top of the inventive enteric drug film coating.

Typically, subcoatings act to separate substances (i.e., APIs) in the core from substances in the inventive enteric drug film coating which may be incompatible with each other. Subcoatings do not influence API release characteristics.

Preferably, any subcoating used with the inventive enteric drug film coating is water-soluble and very thin (i.e., no more than 15 μm, preferably no more than 10 μm in thickness). An exemplary subcoating is hydroxypropylmethyl-cellulose.

A top coating can be applied to the solid dosage form to provide color, a glossy finish, slip, identifying markings (i.e., dosage information, branding, etc.), and/or a protective coating against environmental conditions such as humidity during storage. Top coatings are non-functional and do not influence API release characteristics. Preferably, top coatings are water-soluble.

### DEFINITIONS

Unless stated to the contrary, implicit from the context, or customary in the art, all parts and percents are based on weight, and all test methods are current as of the filing date of this disclosure. For purposes of United States patent practice, the contents of any referenced patent, patent application or publication are incorporated by reference, in their entirety (or its equivalent US version is so incorporated by reference), especially with respect to the disclosure of definitions (to the extent not inconsistent with any definitions specifically provided in this disclosure) and general knowledge in the art.

As used herein, the term “API release value” refers to the percent by weight of the API released from a solid dosage form based on the total weight of the API contained in the core of the solid dosage form. The conditions (i.e., pH, alcohol content, time) during which API is released from a solid dosage form may vary.

“Comprising”, “including”, “having” and like terms are not intended to exclude the presence of any additional component, step or procedure, whether or not the same is specifically disclosed. In order to avoid any doubt, all processes claimed through use of the term “comprising” may include one or more additional steps, pieces of equipment or component parts, and/or materials unless stated to the contrary. In contrast, the term, “consisting essentially of” excludes from the scope of any succeeding recitation any other component, step or procedure, excepting those that are not essential to operability. The term “consisting of” excludes any component, step or procedure not specifically delineated or listed. The term “or,” unless stated otherwise, refers to the listed members individually as well as in any combination.

As used herein, the term “coating weight gain” refers to the percent by weight increase of a solid dosage form resulting from the application of an enteric drug film coating.

### Test Methods

The amount of API release is measured by dissolution testing of samples using, for example, high pressure liquid chromatography (HPLC), ultraviolet-visible (UV-Vis) spectroscopy, or mass spectroscopy in combination HPLC, according to known methods in the art.

Viscosity of the aqueous solutions (e.g., enteric drug film coating, alginic acid salt solution, aqueous shellac component) is determined by use of a Brookfield Viscometer, according to known methods in the art. Viscosity can be expressed as centipoise (cps).

Coating weight gain (thickness) of the enteric drug film coating over the solid dosage form is determined by measuring the weight of the solid dosage form before and after the application of the coating composition, and measuring the percent by weight increase.

### EXPERIMENTAL

Solid dosage tablets containing 2% by weight Red 40 dye are made with 97% by weight spray dried microcrystalline and lactose (MicroLac) and 1% by weight magnesium stearate. The powder blend is compressed into standard round cup 400 mg tablets with 7-10 kilopond (kp) (68.6-98.1 N) tablet hardness.

Enteric drug film coating compositions comprising an aqueous solution of sodium alginate and detackifier (stearic acid), and an aqueous solution of an ammonium salt of shellac, surfactant (polysorbate 80) and plasticizer (glycerin, acetylated monoglyceride), are prepared for each of Examples 1-4 as described below. The aqueous shellac solution is prepared according to Example 5 below.

### Example 1

6 g sodium alginate containing 0.05% by weight stearic acid is added sparingly to 114 g of stirred water in a vortex and continuously stirred for a minimum of 30 minutes until no undispersed sodium alginate is observed 80 g of the aqueous shellac solution from Example 5 is added to the sodium alginate solution and stirred for 5 minutes.

### Example 2

3.1 g sodium alginate containing 0.05% by weight stearic acid is added sparingly to 152 g of stirred water in a vortex and continuously stirred for a minimum of 30 minutes until no undispersed sodium alginate is observed. 43 g of the aqueous shellac solution (Ex. 5) is added to the sodium alginate solution and stirred for 5 minutes.

### Example 3

6 g of sodium alginate containing 0.05% by weight stearic acid is added sparingly to 184 g of stirred water in a vortex with and continuously stirred for a minimum of 30 minutes until no undispersed sodium alginate is observed. 10 g of the aqueous shellac solution (Ex. 5) is added to the sodium alginate solution and stirred for 5 minutes.

### Example 4

7 g of sodium alginate containing 0.05% by weight stearic acid is added sparingly to 112 g of stirred water in a vortex and continuously stirred for a minimum of 30 minutes until no undispersed sodium alginate is observed. 80 g of the aqueous shellac solution (Ex. 5) is added to the sodium alginate solution and stirred for 5 minutes.

### Example 5

An aqueous shellac solution is prepared by mixing together the components listed in Table 1 below.

TABLE 1

| Material | Amount (wt %) |
|---|---|
| Aqueous Shellac (ammonium salt), 25% solution | 89.3 |
| Ethanol | 7.0 |
| Glycerin | 1.8 |
| Distilled Acetylated Monoglycerides | 1.8 |
| Polysorbate 80 | 0.2 |

The enteric drug film coating compositions are coated onto the solid dosage pressed powder tablets described above, as set forth in Tables 2 and 3 below, using a spray device.

TABLE 2
Coating Processing Conditions

| | | |
|---|---|---|
| Tablet Weight | 200 | g |
| Spray Rate | 2.4 | g/min |
| Atomization Pressure | 19 | psi |
| Pattern Air Pressure | 19 | psi |
| Pan Speed | 14 | rpm |
| Exhaust Temperature | 42° | C. |

TABLE 3
Table 3: Enteric Drug Film Coating Composition for Examples 1-4

| | Sodium | | | | | |
|---|---|---|---|---|---|---|
| | Alginate | | | | | |
| | (wt %) | | | | | |
| | (with | | | | | |
| | 0.05 | Aqueous | | | | % |
| Coating | wt % | Shellac | | Solids | Brookfield | Weight |
| Composition | Stearic | Solution | Water | Content | Viscosity | Gain of |
| Example | Acid) | (wt %) | (wt %) | (wt %) | (cps) | Tablet |
| 1 | 3.00 | 40.00 | 57.00 | 13.0 | 592 | 5 |
| 2 | 1.55 | 22.50 | 75.95 | 7.1 | 44 | 5 |
| 3 | 3.00 | 5.00 | 92.00 | 4.2 | 300 | 5 |
| 4 | 3.50 | 40.00 | 56.50 | 13.5 | 1181 | 5 |

The dissolution profile for the coating compositions of Examples 1-4 is determined by immersing the coated tablets in buffer solutions with no alcohol and a pH of 1.2, 4.5 and 7, and in buffer solutions with 5%, 20% and 40%, by volume, alcohol and a pH of 1.2, 4.5 and 7. The buffer solutions are prepared according to USP 36 formulas. The dissolution test is performed as described in USP 36 Method 711 Method II with paddle at 50 rpm in 900 ml of buffer solution.

The weight percent of Red 40 dye released from the tablets at 120 minutes in a buffer solution with a pH of 1.2 (0% alcohol, 40% by volume alcohol) is shown in Table 4. All results showed less than 10% by weight of the Red 40 dye release after 120 minutes.

TABLE 4
Dissolution Results at pH 1, 120 minutes

| | Release of Red 40 dye | Release of Red 40 dye |
|---|---|---|
| Coating | (% by wt) in 0.1N HCl | (% by wt) in 40% by volume |
| Example | (pH = 1.2) | Ethanol, 0.1N HCl (pH 1.2) |
| 1 | 1.1 | 2.3 |
| 2 | 0.0 | 3.9 |
| 3 | 1.3 | 0 |
| 4 | 1.1 | 1.3 |

The alcohol resistant, enteric drug film coating of Example 4 is further tested by immersing coated tablets with a 5% weight gain in a buffer solution at a pH of 4.5 and 7 with 5%, 20%, and 40%, by volume of alcohol under the same dissolution testing conditions set forth above. The results are shown in Tables 5-7, below.

TABLE 5
Dissolution Results for Example 4
Buffer with 0 to 40% by volume EtOH at pH 1.2

| | | Release of Red 40 dye (% by wt) | | | |
|---|---|---|---|---|---|
| | | | | EtOH | EtOH |
| Time | Coating | EtOH | EtOH | 20% | 40% |
| (minutes) | (% wt gain) | 0% by vol. | 5% by vol. | by vol.. | by vol.. |
| 5 | 5 | −0.1 | 0.0 | 0.0 | 0.0 |
| 15 | 5 | 0.7 | 0.4 | −0.1 | 1.6 |
| 30 | 5 | −0.1 | 0.6 | −0.6 | 1.2 |
| 45 | 5 | 0.9 | −1.0 | 0.0 | 0.7 |
| 60 | 5 | −0.1 | −0.2 | −0.9 | 1.2 |
| 75 | 5 | 0.3 | −0.6 | −0.1 | 1.1 |
| 90 | 5 | 0.2 | −0.5 | −1.0 | 0.3 |
| 105 | 5 | 0.6 | −0.6 | −0.7 | 1.4 |
| 120 | 5 | 1.1 | −1.1 | −1.1 | 1.6 |
| 135 | 5 | 1.2 | −0.9 | −0.1 | 1.7 |

TABLE 6
Dissolution Results for Example 4
Buffer with 0 to 40% by volume EtOH at pH 4.5

| | | Release of Red 40 dye (% by wt) | | | |
|---|---|---|---|---|---|
| | | | | EtOH | EtOH |
| Time | Coating | EtOH | EtOH | 20% | 40% |
| (minutes) | (% wt gain) | 0% by vol. | 5% by vol. | by vol. | by vol. |
| 5 | 5 | 0.9 | 0.2 | 0.0 | 0.0 |
| 15 | 5 | 6.9 | 49.6 | 0.0 | 0.0 |
| 30 | 5 | 47.0 | 86.5 | 1.3 | −0.1 |
| 45 | 5 | 84.0 | 99.1 | 19.1 | 1.9 |
| 60 | 5 | 101.4 | 103.6 | 33.1 | 0.3 |
| 75 | 5 | 102.8 | 107.6 | 45.9 | 0.1 |
| 90 | 5 | 102.8 | 110.0 | 54.6 | −0.1 |
| 105 | 5 | 103.1 | 111.4 | 61.7 | 0.0 |
| 120 | 5 | 102.9 | 110.6 | 64.7 | 0.6 |
| 135 | 5 | 102.9 | 110.1 | 67.3 | 0.4 |

TABLE 7
Dissolution Results for Example 4
Buffer with 0 to 40% by volume EtOH at pH 7.0

| | | Release of Red 40 dye (% by wt) | | | |
|---|---|---|---|---|---|
| | | | | EtOH | EtOH |
| Time | Coating | EtOH | EtOH | 20% | 40% |
| minutes | (% wt gain) | 0% by vol. | 5% by vol. | by vol. | by vol. |
| 5 | 5 | 6.9 | 4.2 | 0.0 | −0.9 |
| 15 | 5 | 74.3 | 35.2 | 2.9 | 1.3 |
| 30 | 5 | 87.9 | 59.6 | 17.6 | 1.6 |
| 45 | 5 | 93.6 | 69.4 | 31.4 | 2.4 |
| 60 | 5 | 94.7 | 82.8 | 43.7 | 3.1 |
| 75 | 5 | 94.2 | 92.4 | 55.5 | 4.1 |
| 90 | 5 | 94.4 | 98.7 | 68.3 | 4.9 |
| 105 | 5 | 94.8 | 100.8 | 79.8 | 6.1 |
| 120 | 5 | 94.9 | 103.4 | 87.4 | 7.6 |
| 135 | 5 | 94.9 | 103.8 | 91.0 | 8.8 |

The % by weight release of the API (i.e., Red 40 dye) is related to the API loading and typically based on the average API content of a set number of tablets. Depending on the detection method and content uniformity of the tablets, a variance of +/−10% by weight is generally acceptable under FDA regulations. Thus, the variability of the API in an individual tablet that is tested can result in a % by weight API release value at a negative value (e.g., −0.9% by weight at 5 minutes in Table 7 above) or at a value that is greater than 100% by weight (e.g., as in Table 8).

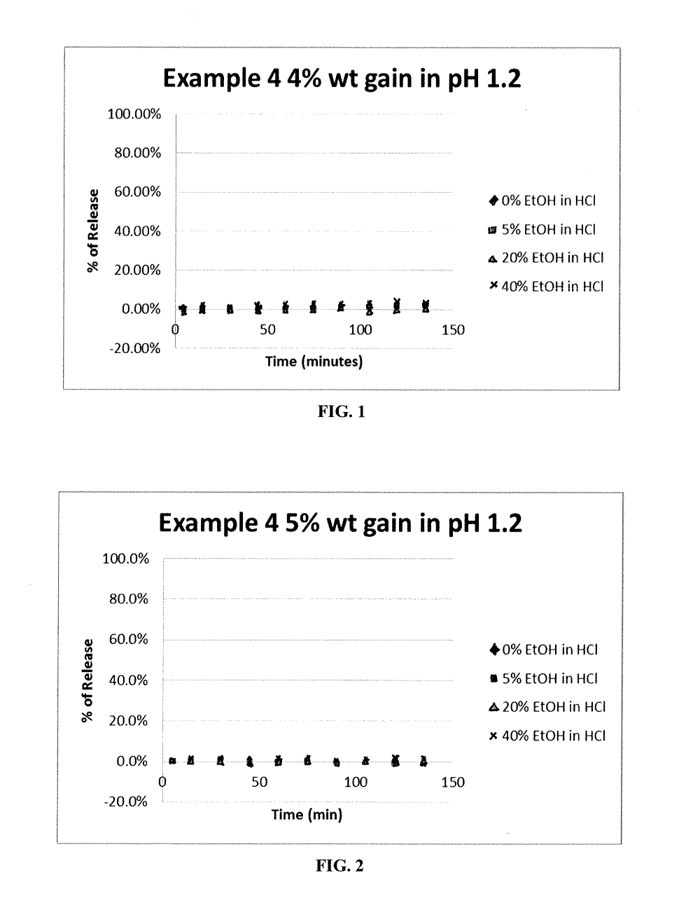

FIGS. 1-6 graphically display the information provided in Tables 5-7, above. As shown by FIGS. 1 and 2, there is very little API (i.e., Red 40 dye) released by the solid dosage tablets (i.e., no rupture/disintegration of the enteric drug film coating) at a pH of 1.2, regardless of the alcohol content of the buffer. This result is consistent with the requirements of enteric drug film coatings which are specifically designed to prohibit release of an API in the stomach (i.e., pH of 1.2).

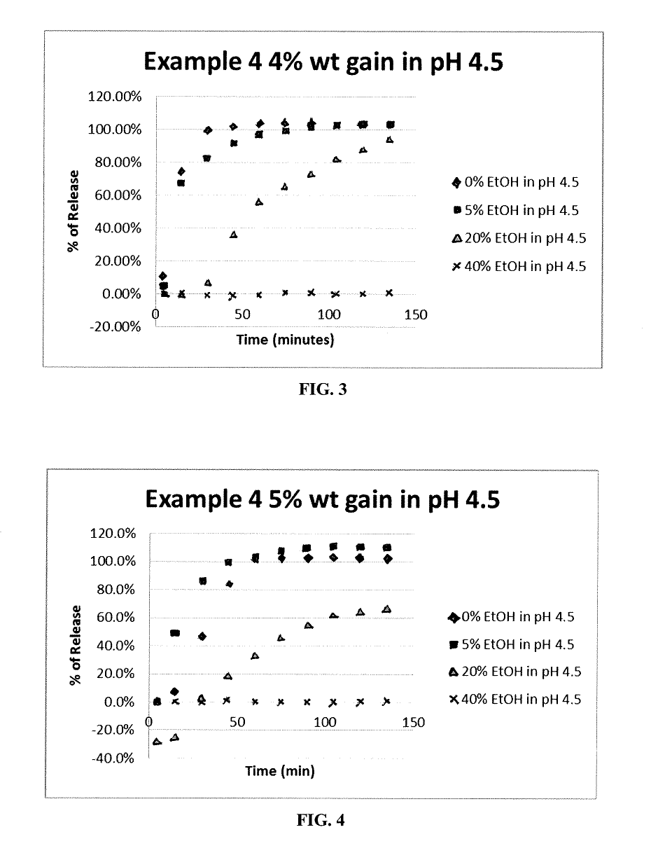

FIGS. 3 and 4 show total API release over time at a pH of 4.5 for the various alcohol concentrations. As illustrated by FIGS. 3 and 4, when there is a low amount of alcohol present (i.e., 20% by volume ethanol or less), the enteric drug film coating ruptures and/or disintegrates as indicated by a majority of the API releasing from the solid dosage tablets within 150 minutes. However, once the amount of alcohol exceeds 25% by volume ethanol (i.e., 40% by volume ethanol), less than 20% by weight of the API (i.e., dye) is released, indicating that rupture and/or disintegration of the enteric drug film coating is inhibited. Specifically, as shown in FIGS. 3 and 4, the present inventive enteric drug film coating prohibits more than 10%, and more than 5%, by weight, API from being released from the solid dosage tablet.

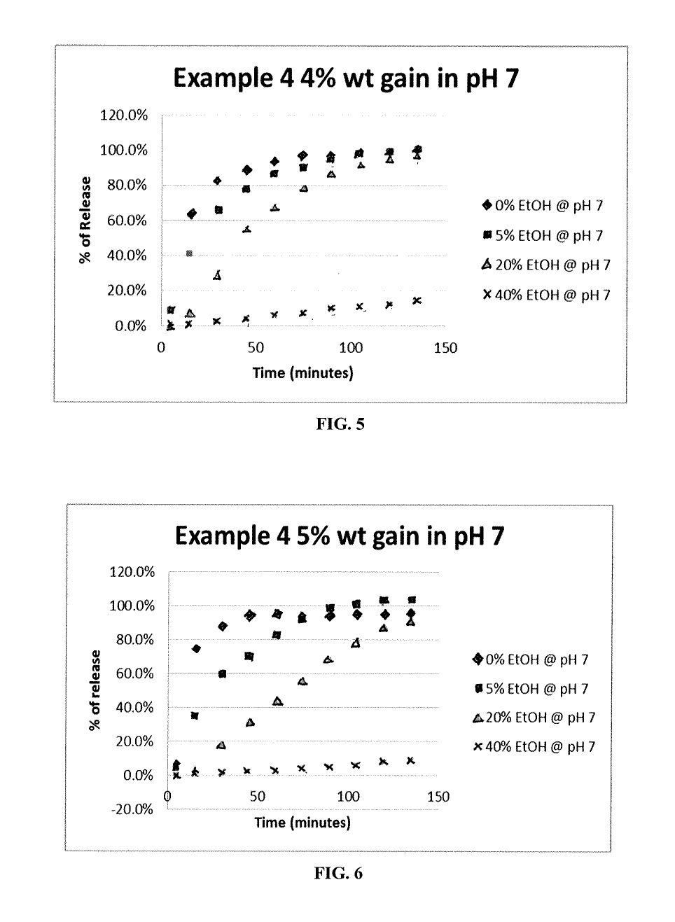

FIGS. 5 and 6 show total API (i.e., dye) release over time in a buffer at a pH of 7.0 for the various alcohol concentrations. Like the embodiments shown in FIGS. 3 and 4, a majority of the API is released from the solid dosage tablets after 150 minutes when the alcohol concentration of the buffer is low (i.e., 20% by volume ethanol or less), indicating that the enteric drug film coating has ruptured and/or disintegrated. In contrast, less than 20% by weight of the API (i.e., dye) is released when the amount of alcohol in the buffer exceeds 25% by volume ethanol (i.e., 40% by volume ethanol), indicating that the enteric drug film coating remains primarily intact (i.e., no disintegration/rupture).

The prevention of dosage dumping observed at 40% by volume ethanol (pH of 4.5 and 7.0) is surprising and unexpected because shellac, a primary component of the inventive enteric drug film coating, is soluble in ethanol. It is surprising that an aqueous solution comprising 5% or less by weight salt of alginic acid and an aqueous shellac solution comprising 10 to 50% by weight of an alkali salt of shellac effectively forms a resultant enteric drug film coating which is alcohol resistant.

A comparison of FIGS. 5 and 6 to FIGS. 3 and 4 does, however, show an increase in API released in a buffer comprising 40% by volume ethanol at a pH of 7.0 as compared to the release in a buffer comprising 40% by volume ethanol at a pH of 4.5. While not being bound to any particular theory, it is thought that the slight increase in release observed at a pH of 7.0 is due to an increased solubility of the shellac in neutral and alkaline solutions. However, as illustrated in FIGS. 5 and 6, the addition of 5% or less by weight of salt of alginic acid to the enteric drug film coating composition comprising an aqueous shellac component still results in an alcohol resistant enteric drug film coating having a total API release of 20% by weight or less at a pH of 7.0.

Since alcohol is also absorbed in the GI tract, a bio-simulation based on the alcohol absorption rate and pH is also conducted to simulate the physiological pH in relation with alcohol. The dissolution test using the tablets coated with the coating composition of Example 4, is run for 135 minutes in a buffer solution with a pH of 1.2 and 40% by volume alcohol. The same tablets are then transferred to a buffer solution with a pH of 4.5 and 30% by volume alcohol for 120 minutes, and then again to a buffer solution with a pH of 7.0 and 5% by volume alcohol for 135 minutes.

The bio-simulation results are shown in Table 8, below.

TABLE 8
Bio-simulation 1 Results

| | API release | | EtOH |
|---|---|---|---|
| Time (min) | (% by wt) | pH | (% by volume) |
| 5 | 0.0 | 1.2 | 40 |
| 15 | −0.1 | 1.2 | 40 |
| 30 | 0.8 | 1.2 | 40 |
| 45 | 0.4 | 1.2 | 40 |
| 60 | 1.8 | 1.2 | 40 |
| 75 | 1.2 | 1.2 | 40 |
| 90 | 1.8 | 1.2 | 40 |
| 105 | 1.7 | 1.2 | 40 |
| 120 | 2.1 | 1.2 | 40 |
| 135 | 2.3 | 1.2 | 40 |
| 140 | 0.0 | 4.5 | 30 |
| 155 | −0.5 | 4.5 | 30 |
| 170 | 0.4 | 4.5 | 30 |
| 185 | −0.6 | 4.5 | 30 |
| 200 | 0.0 | 4.5 | 30 |
| 215 | −0.4 | 4.5 | 30 |
| 230 | 0.7 | 4.5 | 30 |
| 245 | 1.3 | 4.5 | 30 |
| 260 | 0.2 | 4.5 | 30 |
| 275 | 0.4 | 4.5 | 30 |
| 280 | 14.4 | 7.0 | 5 |
| 295 | 34.2 | 7.0 | 5 |
| 310 | 84.3 | 7.0 | 5 |
| 325 | 96.3 | 7.0 | 5 |
| 340 | 98.7 | 7.0 | 5 |
| 355 | 100.7 | 7.0 | 5 |
| 370 | 100.4 | 7.0 | 5 |
| 385 | 100.1 | 7.0 | 5 |
| 400 | 100.6 | 7.0 | 5 |
| 415 | 99.9 | 7.0 | 5 |

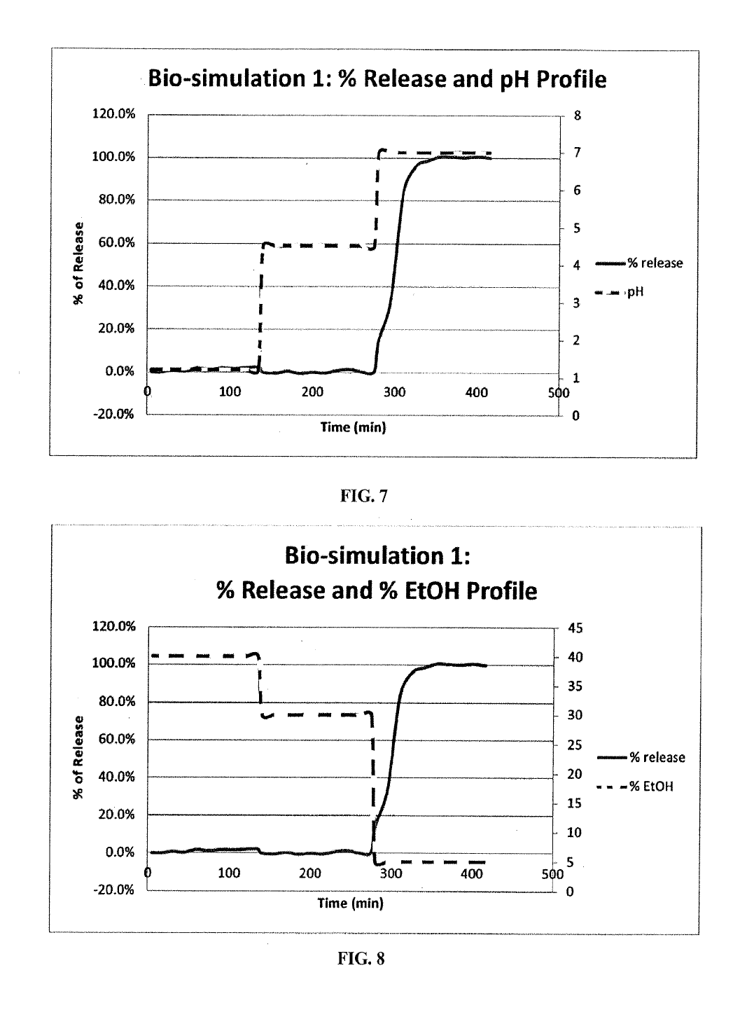

FIGS. 7 and 8 graphically display the information provided in Table 8. FIG. 7 shows the API release over time overlapped with the pH profile of bio-simulation 1. FIG. 8 shows API release over time overlapped with the ethanol content profile of bio-simulation 1.

As shown in FIGS. 7 and 8, the release of a majority of the API (i.e., red dye) is inhibited until the last phase of the simulation (i.e., pH of 7, 5% by volume ethanol). This demonstrates that the inventive enteric drug film coating remains intact (i.e., effectively limited release of the API) at a pH of 1.2 (simulating stomach conditions) and a pH of 4.5 at an alcohol content of 30% by volume ethanol (simulating a high alcohol content in the small intestines). Once the alcohol content decreased to below 25% by volume ethanol, or a majority of the alcohol consumed is absorbed, the enteric drug film coating ruptures and/or disintegrates and API is released.

A second bio-simulation is performed using the tablets coated with the coating composition of Example 4. In the second bio-simulation, the dissolution test is run for 135 minutes in a buffer solution with a pH of 1.2 and 40% by volume alcohol. The same tablets are transferred to a buffer solution with a pH of 4.5 and 30% by volume alcohol for 60 minutes, and then again to a buffer solution with a pH of 4.5 and 20% by volume alcohol for 60 minutes.

The results of the second bio-simulation are shown in Table 9, below.

TABLE 9
Bio-simulation 2 Results

| | API release | | EtOH |
|---|---|---|---|
| Time (min) | (% by wt) | pH | (% by volume) |
| 5 | 0.2 | 1.2 | 40 |
| 15 | 1.5 | 1.2 | 40 |
| 30 | 2.9 | 1.2 | 40 |
| 45 | 0.7 | 1.2 | 40 |
| 60 | −0.4 | 1.2 | 40 |
| 75 | 0.6 | 1.2 | 40 |
| 90 | 2.0 | 1.2 | 40 |
| 105 | 1.8 | 1.2 | 40 |
| 120 | 1.4 | 1.2 | 40 |
| 135 | 2.9 | 1.2 | 40 |
| 140 | 0.0 | 4.5 | 30 |
| 155 | 0.2 | 4.5 | 30 |
| 170 | −0.2 | 4.5 | 30 |
| 185 | 0.8 | 4.5 | 30 |
| 200 | 1.1 | 4.5 | 30 |
| 215 | 0.0 | 4.5 | 20 |
| 230 | 6.4 | 4.5 | 20 |
| 245 | 28.9 | 4.5 | 20 |
| 260 | 40.6 | 4.5 | 20 |
| 275 | 50.2 | 4.5 | 20 |

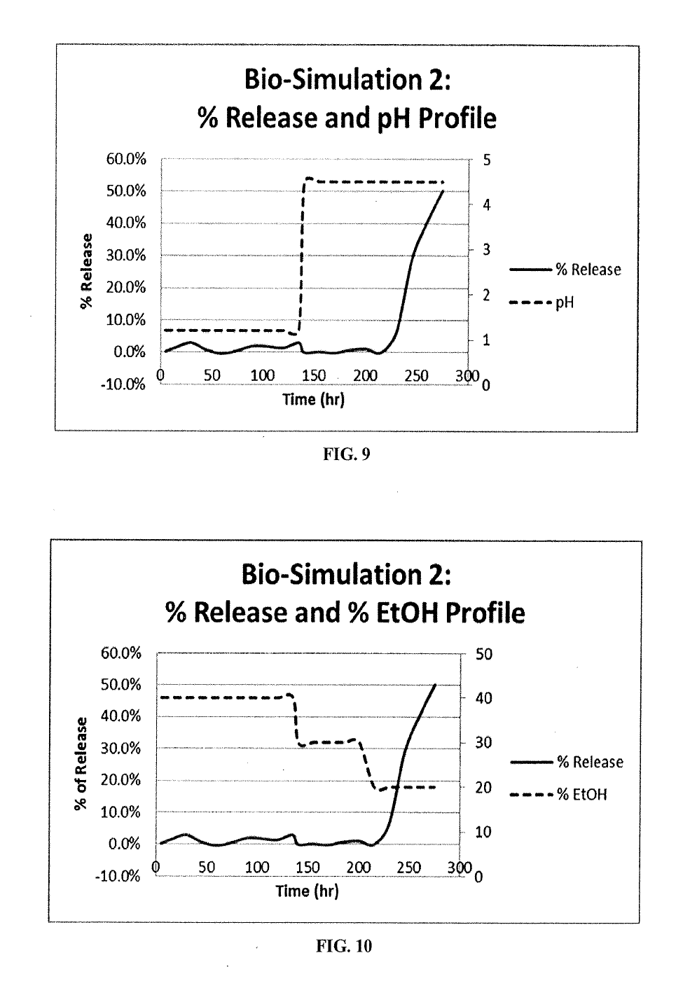

FIGS. 9 and 10 graphically display the information provided in Table 9. FIG. 9 shows the API release over time overlapped with the pH profile of bio-simulation 1. FIG. 10 shows API release over time overlapped with the ethanol content profile of bio-simulation 1.

As shown in FIGS. 9 and 10, the release of a majority of the API (i.e., red dye) is inhibited until the last phase of the simulation (i.e., pH of 4.5, 20% by volume ethanol). This demonstrates that the inventive enteric drug film coating remains intact (i.e., effectively limited release of the API) at a pH of 1.2 (simulating stomach conditions) and a pH of 4.5 at an alcohol content of 30% by volume ethanol (simulating a high alcohol content in the small intestines). Once the alcohol content decreased to below 25% by volume ethanol, or a majority of the alcohol consumed is absorbed, the enteric drug film coating ruptures/disintegrates and the API (i.e., red dye) is released.

Although the invention is described herein with reference to specific embodiments, it will be appreciated by those of ordinary skill in the art that various modifications and changes can be made without departing from the scope of the present disclosure as set forth in the claims below. Accordingly, the specification is to be regarded in an illustrative rather than a restrictive sense, and all such modifications are intended to be included within the scope of the present disclosure. Any benefits, advantages, or solutions to problems that are described herein with regard to specific embodiments are not intended to be construed as a critical, required or essential feature or element of any or all of the claims. For the purposes of United States patent practice, the contents of any referenced patent, patent application or publication are incorporated by reference in their entirety.

1. An enteric drug film coating composition, comprising an aqueous solution comprising:
1-5% by weight of a salt of alginic acid; and
an aqueous shellac component;
the % by weight based on the total weight of the composition.

2. The enteric drug film coating composition of claim 1, comprising 3-4% by weight of the salt of alginic acid.

3. The enteric drug film coating composition of claim 1, wherein the salt of alginic acid is selected from the group consisting of sodium alginate, potassium alginate, and combinations thereof.

4. The enteric drug film coating composition of claim 3, wherein the salt of alginic acid is sodium alginate.

5. The enteric drug film coating composition of claim 4, wherein a 1% aqueous solution of the sodium alginate has a Brookfield viscosity at ambient temperature of less than 6 cps.

6. The enteric drug film coating composition of claim 1, wherein the aqueous shellac component comprises an alkali salt of shellac.

7. The enteric drug film coating composition of claim 6, wherein the alkali salt of shellac comprises an ammonium salt of shellac.

8. The enteric drug film coating composition of claim 1, further comprising at least one additive selected from the group consisting of a detackifier, slip aid, surfactant, plasticizer, preservative, opacifier, colorant, and combinations thereof.

9. The enteric drug film coating composition of claim 8, wherein the detackifier is selected from the group consisting of silicon dioxide, stearic acid, salt of a fatty acid, talc, calcium chloride, dicalcium phosphate, starch, maltodextrin, lactose, microcrystalline cellulose, mannitol, xylitol, sorbitol, maltitol, and combinations thereof.

10. The enteric drug film coating composition of claim 8, wherein the surfactant is selected from the group consisting of lecithin, polysorbates, glycerol esters of fatty acids, sugar esters of fatty acids, and combinations thereof.

11. The enteric drug film coating composition of claim 8, wherein the plasticizer is selected from the group consisting of glycerin, acetylated glyceride, medium chain triglycerides, lecithin, vegetable derived oils, mineral oils, triacetin, triethyl citrate, propylene glycol, polyethylene glycol, dibutyl sebacate, and combinations thereof.

12. The enteric drug film coating composition of claim 8, wherein the opacifier is selected from the group consisting of titanium dioxide, zinc oxide, calcium diphosphate, and combinations thereof.

13. The enteric drug film coating composition of claim 8, wherein the colorant is selected from the group consisting of synthetic dyes, lakes, natural dyes, oxide colors, and combinations thereof.

14. The enteric drug film coating composition of claim 8, wherein the preservative is selected from the group consisting of a salt of sorbic acid, a salt of benzoic acid, methyl parabens, propyl parabens, and combinations thereof.

15. An enteric drug film coating comprising the enteric drug film coating composition of claim 1, wherein the enteric drug film coating is alcohol resistant.

16. An oral solid dosage form having a controlled release profile for release of an API within the intestinal tract, the solid dosage form comprising:
a core comprising the API; and
a coating comprising the enteric drug film coating of claim 15.

17. The solid dosage form of claim 16, having a coating weight gain of 1-10% by weight of the total weight of the solid dosage form.

18. The solid dosage form of claim 16, wherein the enteric film coating has an API release value of 30% by weight or less of the total weight of the API of the solid dosage form after exposure to a pH of 1.2 and an alcohol content of greater than 25% by volume ethanol for up to 120 minutes.

19. The solid dosage form of claim 18, wherein the API release value is 20% by weight or less.

20. The solid dosage form of claim 19, wherein the pH is 4.5 and the alcohol content is greater than or equal to 30% by volume ethanol.

21-26. (canceled)

27. A method of preparing an oral solid dosage form, comprising:
applying a enteric drug film coating comprising the enteric drug film coating composition of claim 1 onto a core comprising an API, wherein the enteric drug film coating is alcohol resistant.

28. The method of claim 27, wherein the enteric drug film coating is effective to inhibit dose dumping at a high alcohol concentration in the gastrointestinal tract within a body.

29. The method of claim 28, wherein the alcohol concentration is greater than 5% by volume.

30. The method of claim 28, wherein the enteric drug film coating is effective to inhibit dose dumping at a pH greater than 1.2.
